(19) United States
(12) Patent Application Publication
(10) Pub. No.: US 2026/0188501 A1
(43) Pub. Date: Jul. 2, 2026

(54) Hardware-Anchored Autonomous Deployment, Shadow Execution, and Sub-Millisecond Rollback System for Localized Predetermined Change Control Plans

(71) Applicant: George William Bickerstaff, III, Greenwich, CT (US)

(72) Inventor: George William Bickerstaff, III, Greenwich, CT (US)

(21) Appl. No.: 19/546,366

(22) Filed: Feb. 22, 2026

(51) Int. Cl. G16H 50/20; G16H 15/00

(52) U.S. Cl. CPC G16H 50/20; G16H 15/00

(57) ABSTRACT

A hardware-anchored deployment and safety system for continuous learning artificial intelligence at the local hospital edge. The system safely receives globally approved AI updates and isolates them within an ephemeral sandbox enclave. A hardware bifurcator splits live patient data, subjecting the incoming model to a silent, hardware-enforced shadow execution phase. A local divergence comparator tests the shadow model's performance and forwards metrics to an efficacy threshold gate. The efficacy threshold gate evaluates the metrics against a Predetermined Change Control Plan (PCCP) Manifest stored in a Localized PCCP Vault. If local efficacy is proven, an autonomous transition gate physically swaps the models without diagnostic downtime. A hardwired symbiotic rollback circuit constantly monitors the newly deployed model's live performance, capable of bypassing the local operating system to force a sub-millisecond reversion to a prior algorithmic state if clinical safety thresholds are breached, ensuring zero-risk localization of globally evolving medical software.

### FIELD

The present invention relates to hardware-secured artificial intelligence (AI) lifecycle management at the clinical edge. More particularly, the invention provides a silicon-anchored architecture that receives globally consensus-approved AI updates, tests them silently via hardware-enforced shadow execution against local clinical demographics, and executes autonomous, hardwired version transitions and emergency rollbacks based on a Predetermined Change Control Plan (PCCP) Manifest stored within a Localized PCCP Vault.

While federated networks can safely aggregate and approve global artificial intelligence updates, pushing a global model directly into live clinical diagnostic workflows at a local hospital remains inherently dangerous. A globally optimized AI model may perform poorly when exposed to the specific demographic nuances, hardware calibration quirks, or workflow variations of an individual edge hospital. There is an unmet need for a localized hardware system that autonomously intercepts incoming global models, forces them to prove their efficacy locally via silent shadow execution, and utilizes un-bypassable physical circuits to deploy or rollback the model based on strict regulatory manifests.

### Practical Application & Non-Obviousness

The claimed invention integrates automated software deployment into a specific, hardware-secured analog and digital architecture that materially alters the local edge processor state to execute zero-risk continuous learning. By utilizing hardware-enforced shadow execution and a physically hardwired fallback circuit, this system achieves autonomous, sub-millisecond model rollbacks if localized performance thresholds are breached, as demonstrated in Example 2. This significantly mitigates the catastrophic risks of localized algorithmic drift caused by global updates, providing a quantifiable technological improvement over generic software update mechanisms that satisfies 35 U.S.C. § 101. Furthermore, this hardware integration directly addresses long-felt, unmet regulatory needs for safe local deployment of continuous learning models since the inception of the FDA PCCP framework, providing strong secondary indicia of non-obviousness that explicitly bolsters the Graham factors under 35 U.S.C. § 103.

### Definitions

Autonomous Version Transition Gate: A physical logic circuit that seamlessly swaps a hospital's active diagnostic AI with a newly updated model. It executes the transition in exactly one processor clock cycle to prevent any clinical diagnostic downtime. It physically cannot trigger unless the new model successfully passes all shadow execution testing mandated by the Efficacy Threshold Gate.

Efficacy Threshold Gate: A dedicated logic controller that actively monitors the mathematical outputs recorded in the Shadow Inference Record. It systematically compares the shadow model's localized diagnostic accuracy against the strict safety limits defined in the Localized PCCP Vault. It prevents the untested model from advancing to the Autonomous Version Transition Gate until it mathematically proves its clinical superiority.

Ephemeral Sandbox Enclave: A highly secure, mathematically isolated hardware partition temporarily generated solely to house an incoming global model update. It prevents the untested software from interacting with the hospital's primary diagnostic systems. It automatically destroys itself and its contents if physical tampering is detected.

Hardware-Enforced Shadow Execution: A mandatory testing phase where an updated AI model runs silently in the background alongside the live diagnostic model. It processes identical patient data but is physically blocked from transmitting its predictions to human clinicians. It generates a mathematically pure track record of how the new model would have performed if it were live.

Local Divergence Comparator: A hardware-accelerated analytical tool that continuously compares the live AI model's predictions against the shadow AI model's predictions. It mathematically measures the exact clinical deviation between the older algorithm and the proposed update. It routes this deviation data directly into the Efficacy Threshold Gate for regulatory validation.

Localized PCCP Vault: A hardware-secured memory sector strictly dedicated to storing a digitally signed Predetermined Change Control Plan (PCCP) Manifest. The stored PCCP Manifest dictates the maximum acceptable performance drop a global model can exhibit when applied to local patient demographics. The vault acts as the ultimate physical legal barrier preventing unsafe software updates from going live.

Primary Execution State: The designated active hardware pathway through which approved medical diagnostic models evaluate patient data. Any artificial intelligence operating within this state is granted physical permission to transmit results to a doctor's screen. Only one model version may occupy this state at any given moment.

Shadow Inference Record: An append-only, cryptographic ledger that permanently documents the background performance of an untested AI model. It provides indisputable mathematical proof of a new model's local efficacy before a hospital adopts it. It serves as essential regulatory evidence for federal continuous learning audits.

Sub-Millisecond State Reversion: The emergency physical process of immediately disconnecting a failing AI model from the primary execution state. It simultaneously reconnects the prior, stable algorithm to the live clinical data stream. It relies entirely on hardwired circuitry to bypass standard operating systems and achieve near-zero latency.

Symbiotic Rollback Circuit: A dedicated, physical fail-safe wire built directly into the local edge processor motherboard. It constantly receives real-world performance metrics from the active medical artificial intelligence. It automatically forces a sub-millisecond state reversion if those metrics breach the parameters defined by the PCCP Manifest stored in the Localized PCCP Vault.

### BRIEF DESCRIPTION OF THE DRAWINGS

Referring now to the drawings submitted separately in compliance with 37 CFR 1.84, the following figures illustrate the preferred embodiments of the invention.

### DETAILED DESCRIPTION OF THE DRAWINGS

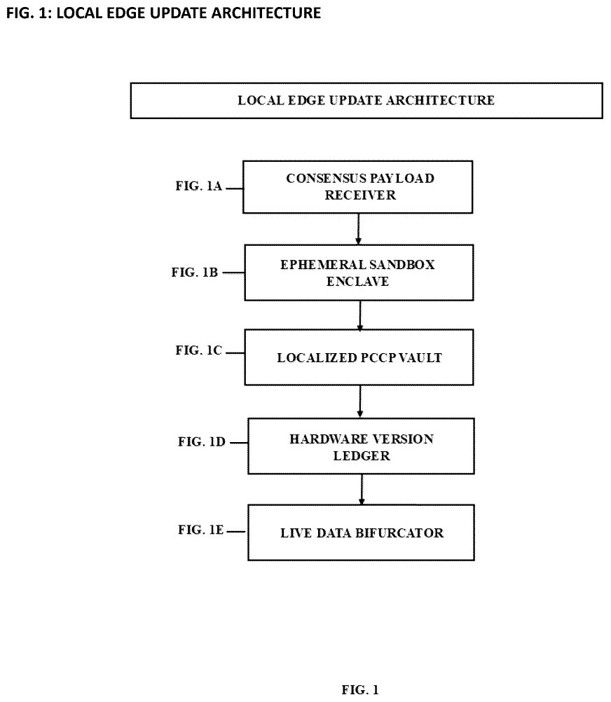

### FIG. 1: Local Edge Update Architecture

Referring now to FIG. 1A, the Consensus Payload Receiver acts as the secure digital docking bay for incoming global artificial intelligence updates. It strictly accepts model parameters that carry a verified mathematical signature from a distributed federated network. It physically drops any software packages originating from unauthorized or unverified external sources.

Referring now to FIG. 1B, the Ephemeral Sandbox Enclave creates a temporarily isolated execution space exclusively for the incoming global model. It mathematically isolates the newly arrived algorithms from the hospital's live diagnostic pipeline. This physical separation ensures that untested software cannot accidentally interact with live patient data.

Referring now to FIG. 1C, the Localized PCCP Vault securely stores the digitally signed PCCP Manifest governing the local hospital node. It utilizes tamper-responsive silicon memory to protect the hospital's unique demographic safety boundaries. It physically prevents local hospital administrators from manually overriding federal medical device regulations.

Referring now to FIG. 1D, the Hardware Version Ledger permanently records the specific history of artificial intelligence models deployed at the facility. It logs exactly when a model was received, tested, deployed, or rolled back. It provides a flawless, unalterable digital timeline for internal hospital compliance officers.

Referring now to FIG. 1E, the Live Data Bifurcator safely splits the incoming stream of real-time patient medical information. It sends one identical data copy to the live diagnostic model and another to the isolated sandbox. It guarantees that both models are evaluated using the exact same pristine clinical evidence.

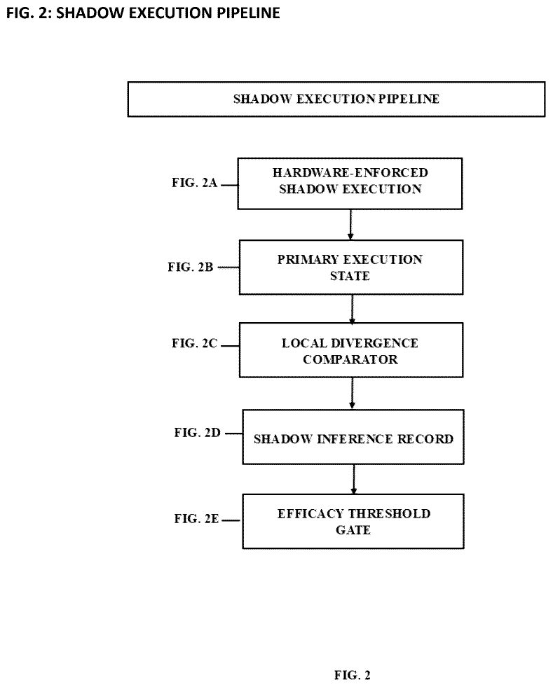

### FIG. 2: Shadow Execution Pipeline

Referring now to FIG. 2A, the Hardware-Enforced Shadow Execution module silently runs the newly received global model in the background. It physically blocks the new model's predictions from ever reaching a human doctor's visual interface. It generates thousands of test diagnoses completely invisibly to the hospital's active medical staff.

Referring now to FIG. 2B, the Primary Execution State simultaneously processes the exact same patient data using the older, trusted model. It retains exclusive physical authorization to push its medical recommendations to the clinical staff. It ensures absolute diagnostic stability while the new software undergoes its rigorous background testing.

Referring now to FIG. 2C, the Local Divergence Comparator continuously analyzes the mathematical outputs from both the shadow and live models. It calculates precisely how the proposed global update alters the diagnostic outcomes for the local patient population. It quantifies these algorithmic differences using strict statistical significance testing and forwards the results to the Efficacy Threshold Gate.

Referring now to FIG. 2D, the Shadow Inference Record permanently logs every single background prediction made by the untested model. It creates a mathematically secure chain of evidence detailing the new software's exact local performance. It generates an unforgeable testing history required for subsequent deployment authorization.

Referring now to FIG. 2E, the Efficacy Threshold Gate actively monitors the accumulated data inside the shadow inference record. It compares the shadow model's localized accuracy against the safety limits defined in the PCCP Manifest stored in the Localized PCCP Vault. It keeps the model trapped in the shadow phase until it mathematically proves its clinical superiority.

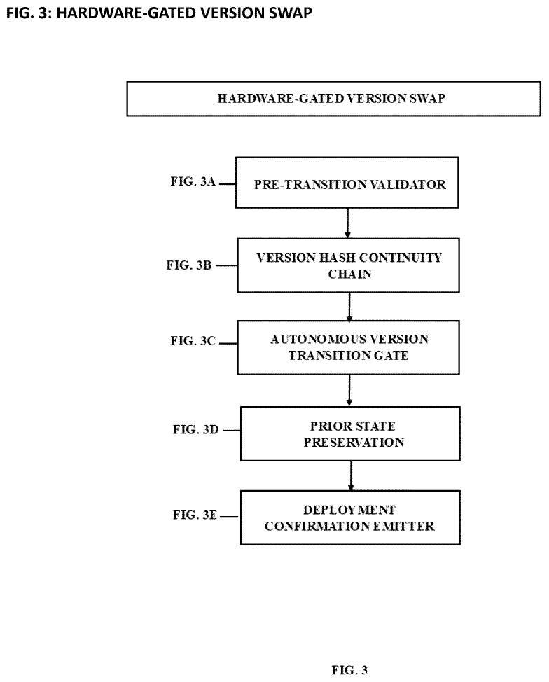

### FIG. 3: Hardware-Gated Version Swap

Referring now to FIG. 3A, the Pre-Transition Validator executes a final security sweep immediately before a software swap is authorized. It mathematically confirms that the Efficacy Threshold Gate has successfully validated all localized regulatory safety thresholds. It physically aborts the deployment sequence if even a single statistical safety metric is failing.

Referring now to FIG. 3B, the Version Hash Continuity Chain generates a cryptographic link between the outgoing model and the incoming model. It securely locks the two software versions together in the hospital's Hardware Version Ledger. It mathematically proves that the facility maintained a perfect, sequential upgrade path.

Referring now to FIG. 3C, the Autonomous Version Transition Gate initiates the physical swap of the artificial intelligence models. It routes the live clinical data stream away from the older algorithm and directly into the newly approved model. It completes this highly complex transition in a single processor clock cycle to prevent clinical downtime.

Referring now to FIG. 3D, the Prior State Preservation module immediately archives the outgoing, older version of the artificial intelligence. It freezes the older model's exact mathematical state inside a highly secure hardware vault. It ensures the stable software remains instantly accessible in case a rapid emergency reversion is required.

Referring now to FIG. 3E, the Deployment Confirmation Emitter broadcasts a secure signal back to the global federated network. It mathematically proves to the central consensus body that the local hospital successfully adopted the new global standard. It allows the broader network to track exactly which facilities are running the latest software.

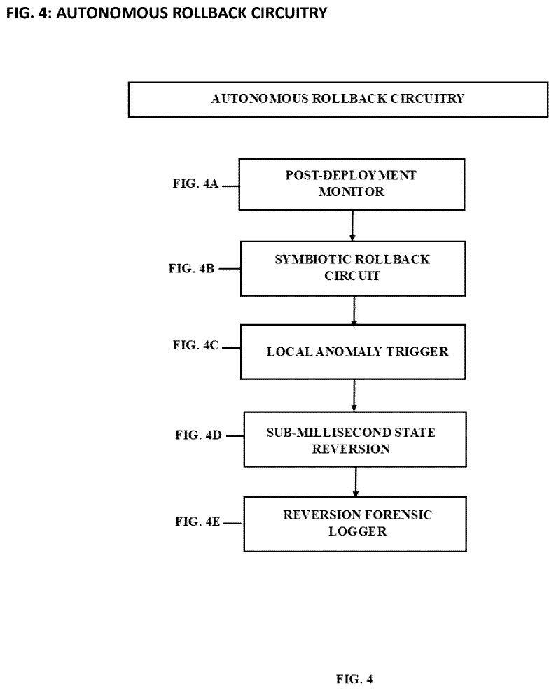

### FIG. 4: Autonomous Rollback Circuitry

Referring now to FIG. 4A, the Post-Deployment Monitor increases its monitoring sensitivity immediately after a new model enters the primary execution state. It continuously scans the newly live algorithm's diagnostic outputs for sudden statistical degradation. It remains in a state of high alert during the crucial first weeks of clinical deployment.

Referring now to FIG. 4B, the Symbiotic Rollback Circuit operates as an entirely independent, hardwired fail-safe mechanism. It physically bypasses the hospital's primary operating system to ensure no software virus can delay its activation. It stands ready to sever the live model's connection to the diagnostic interface at any moment.

Referring now to FIG. 4C, the Local Anomaly Trigger fires instantly if the new model's live performance breaches the boundaries of the PCCP Manifest. It sends an un-blockable electrical signal directly to the autonomous version transition gate. It fundamentally overrides all other administrative software commands present in the system.

Referring now to FIG. 4D, the Sub-Millisecond State Reversion immediately disconnects the failing artificial intelligence model from the primary execution state. It physically routes the live patient data instantly back into the archived, prior version of the model. It completely neutralizes the clinical threat before a human doctor even notices an algorithmic error.

Referring now to FIG. 4E, the Reversion Forensic Logger automatically captures a mathematical snapshot of the exact system state that caused the failure. It securely stores the specific patient data and algorithmic weights that triggered the emergency rollback. It provides human software engineers with the exact digital evidence needed to patch the failed algorithm.

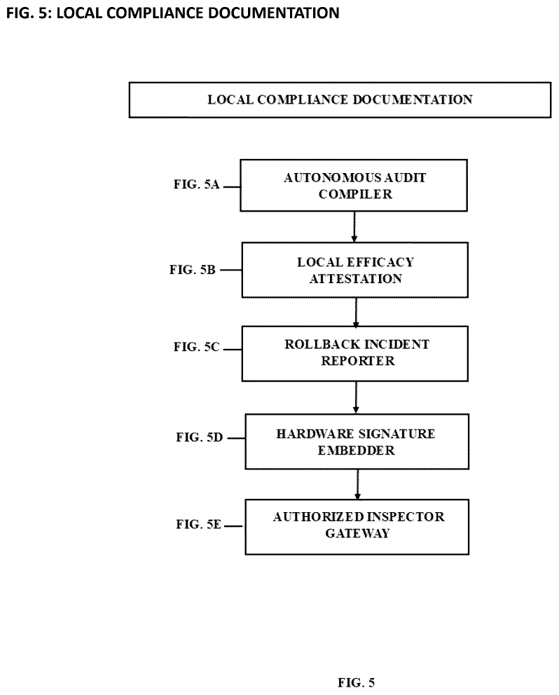

### FIG. 5: Local Compliance Documentation

Referring Now to FIG. 5A, the Autonomous Audit Compiler systematically gathers data from both the shadow testing phase and the live deployment. It translates complex hardware execution logs into standardized, highly readable regulatory reports. It eliminates the need for human compliance officers to manually draft continuous learning documentation.

Referring now to FIG. 5B, the Local Efficacy Attestation mathematically proves exactly why a global update was either accepted or rejected by the hospital. It generates a cryptographically signed certificate detailing the model's exact performance on local patient demographics. It legally defends the hospital's automated software deployment decisions to federal inspectors.

Referring now to FIG. 5C, the Rollback Incident Reporter automatically files emergency documentation if the symbiotic rollback circuit is ever triggered. It strictly formats the failure metrics according to the FDA's rapid adverse event reporting requirements. It ensures the hospital remains legally compliant even during an acute software failure.

Referring now to FIG. 5D, the Hardware Signature Embedder physically brands every compliance report with the hospital edge node's unforgeable identity. It proves that the shadow testing and rollbacks were executed by physical logic gates, not falsified software simulations. It provides absolute mathematical assurance to external healthcare regulatory bodies.

Referring now to FIG. 5E, the Authorized Inspector Gateway provides federal auditors with a highly secure, read-only digital portal. It allows regulators to instantly pull hardware-verified continuous learning records during on-site hospital inspections. It fundamentally streamlines the regulatory oversight of constantly evolving medical artificial intelligence.

### Examples of Enablement

Example 1: Shadow Execution of a Global Sepsis Model. A Local hospital edge node receives a newly consensus-approved global Sepsis prediction model. Instead of deploying it immediately, the Ephemeral Sandbox Enclave initiates Hardware-Enforced Shadow Execution. For 14 days, the Live Data Bifurcator routes real-time ICU patient data to both the live model and the shadow model. The Local Divergence Comparator analyzes the deviation and routes the results to the Efficacy Threshold Gate. The Efficacy Threshold Gate mathematically proves that the new shadow model detects sepsis 4 hours earlier than the live model across the local hospital's specific patient demographic, perfectly satisfying the parameters of the PCCP Manifest stored in the Localized PCCP Vault. The Autonomous Version Transition Gate executes the swap in one clock cycle, safely modernizing the hospital's diagnostic capabilities without exposing patients to untested algorithmic risks.

Example 2: Sub-Millisecond Rollback of a Radiology Algorithm. Following a successful shadow testing phase, an edge node deploys an updated lung nodule detection model to the Primary Execution State. Two weeks later, the hospital recalibrates its physical CT scanners, subtly altering the image contrast of the incoming data. The new AI model struggles with this unexpected local artifact and its false-positive rate suddenly spikes beyond the safety limits defined in the PCCP Manifest. The Symbiotic Rollback Circuit instantly detects the breach and triggers a Sub-Millisecond State Reversion. Bypassing the local operating system, the circuit immediately disconnects the new model and reconnects the prior, stable AI model to the live feed, completely averting a wave of false cancer diagnoses while autonomously generating an FDA Rollback Incident Report.

What is claimed:

1. A hardware-anchored autonomous deployment and shadow execution system for localized medical artificial intelligence, comprising: a secure edge processor; an ephemeral sandbox enclave configured to receive a globally approved artificial intelligence model update; a live data bifurcator hardwired to duplicate real-time clinical data streams;
a localized PCCP vault storing a digitally signed predetermined change control plan manifest; a local divergence comparator configured to execute hardware-enforced shadow execution by comparing shadow model predictions against live model predictions;
an efficacy threshold gate configured to validate predictions generated by the local divergence comparator; and an autonomous version transition gate comprising a physical logic circuit configured to deploy the updated artificial intelligence model to a primary execution state exclusively upon verifying that the shadow execution metrics validated by the efficacy threshold gate mathematically satisfy the predetermined change control plan manifest stored within the localized PCCP vault.

2. A method for the localized testing and zero-downtime deployment of continuous learning artificial intelligence, comprising the steps of:
receiving a consensus-approved global model update at a local hardware edge node; isolating the global model update within an ephemeral sandbox enclave; conducting hardware-enforced shadow execution by evaluating live patient data simultaneously through both an active primary model and the isolated global model update;
mathematically measuring the clinical deviation between the models using a local divergence comparator; passing the measured clinical deviation to an efficacy threshold gate for regulatory validation;
generating an unforgeable shadow inference record; and executing a physical, single-clock-cycle version transition via an autonomous version transition gate to swap the active primary model for the global model update strictly upon cryptographically proving local safety efficacy against a predetermined change control plan manifest stored within a localized PCCP vault.

3. An autonomous fallback and sub-millisecond state reversion architecture for local clinical edge nodes, comprising: a primary execution state executing an active medical artificial intelligence model; a hardware version ledger securely archiving a prior stable version of said artificial intelligence model; and a symbiotic rollback circuit physically hardwired to the primary execution state, wherein the rollback circuit continuously monitors live diagnostic statistical performance and is configured to completely bypass local host operating systems to execute a sub-millisecond state reversion to the archived prior model version if live performance metrics breach hardcoded boundaries defined by a predetermined change control plan manifest stored within a localized PCCP vault.

4. The system of claim 1, wherein the ephemeral sandbox enclave physically blocks the updated artificial intelligence model from transmitting any diagnostic predictions to external clinical interfaces during the hardware-enforced shadow execution phase.

5. The system of claim 1, wherein the efficacy threshold gate automatically destroys the globally approved artificial intelligence model update if the shadow execution metrics violate the predetermined change control plan manifest stored within the localized PCCP vault.

6. The system of claim 1, further comprising a version hash continuity chain configured to cryptographically lock the algorithmic state of the outgoing live model to the incoming updated model inside a hardware version ledger.

7. The method of claim 2, wherein the real-time clinical data stream processed during the hardware-enforced shadow execution phase comprises cryptographically sealed raw sensor data proving an unadulterated point of capture.

8. The method of claim 2, further comprising the step of broadcasting a cryptographically signed deployment confirmation emitter signal back to a global consensus network strictly after the single-clock-cycle version transition is successfully completed.

9. The method of claim 2, wherein the hardware-enforced shadow execution phase evaluates the globally approved model explicitly against the unique, localized demographic traits of the native hospital deployment site.

10. The architecture of claim 3, wherein the sub-millisecond state reversion is executed entirely via combinational hardware logic gates, rendering the emergency rollback completely immune to software-level network latency or administrative privilege escalation attacks.

11. The architecture of claim 3, further comprising a reversion forensic logger configured to instantly capture and cryptographically seal the exact patient data vectors and algorithmic weights present at the exact microsecond the performance breach occurred.

12. The method of claim 2, wherein the requirement for localized shadow execution and hardware-gated version transition directly resolves long-felt regulatory needs by guaranteeing that globally optimized medical algorithms are empirically proven safe on local hospital populations prior to clinical exposure.
